(19) United States
(12) Patent Application Publication
(10) Pub. No.: US 2005/0080255 A1
(43) Pub. Date: Apr. 14, 2005

(54) Crystalline cefdinir potassium dihydrate

(76) Inventors: Yatendra Kumar, Gurgaon (IN); Mohan Prasad, Gurgaon (IN); Ashok Prasad, New Delhi (IN); Shailendra Kumar Singh, Gurgaon (IN); Neela Praveen Kumar, Hyderabad (IN)

Correspondence Address: Jayadeep R Deshmukh;Ranbaxy Inc, Suite 2100, 600 College Road East, Princeton, NJ, 08540, US

(21) Appl. No.: 10/498,406

(22) PCT Filed: Dec. 12, 2002
(86) PCT No.: PCT/IB02/05315

(30) Foreign Application Priority Data
Dec. 13, 2001 (IN) 1242/DEL/2001

(51) Int. Cl. C07D 501/14

(52) U.S. Cl. 540/222

(57) ABSTRACT

The present invention relates to a novel crystalline cefdinir potassium dihydrate, to a process for its preparation and to a method of preparing pure cefdinir via the crystalline salt.

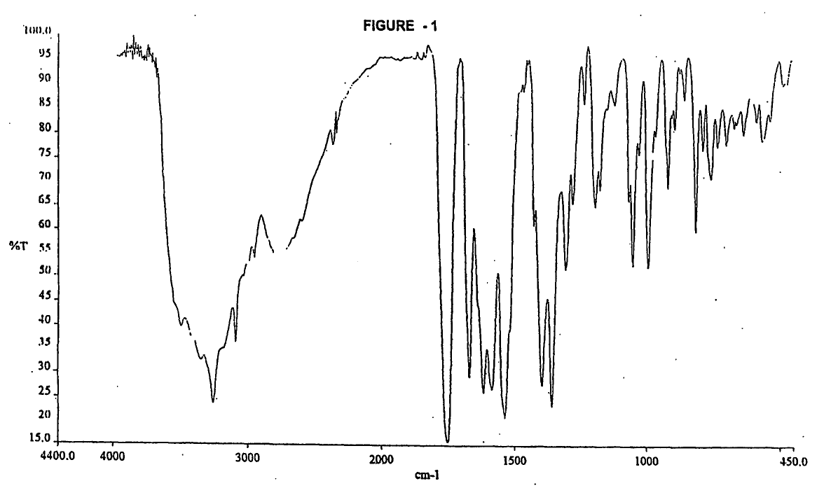

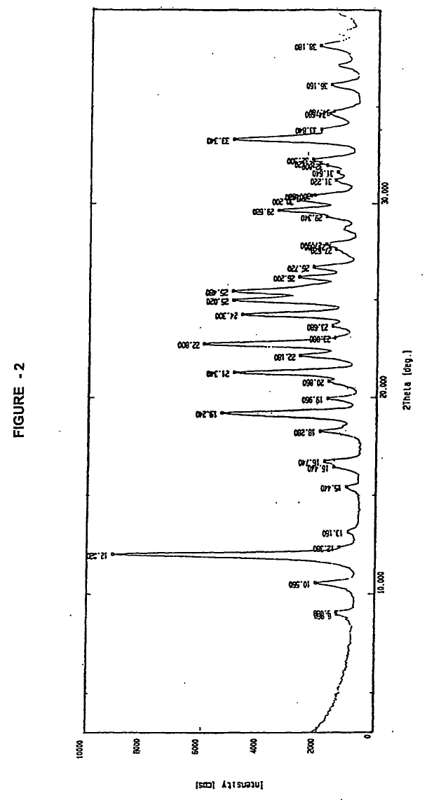

### FIELD OF THE INVENTION

The present invention relates to a novel crystalline cefdinir potassium dihydrate, to a process for its preparation and to a method of preparing pure cefdinir via the crystalline salt.

### BACKGROUND OF THE INVENTION

Cefdinir potassium is chemically known as potassium 7-[2-(2-aminothiazol-4-yl)-2-hydroxyiminoacetamido]-3-vinyl-3-cephem-4-carboxylate (syn isomer) of formula I, 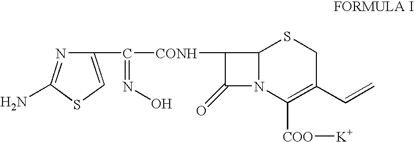
and was described for the first time in U.S. Pat. No. 4,559,334. Cefdinir is a third generation cephalosporin antibiotic for oral administration and has a broader antibacterial spectrum than other orally administrable antibiotics. Cefdinir is particularly effective against staphylococci and streptococci.

U.S. Pat. No. 4,559,334 describes the preparation of cefdinir sodium and its isolation via chromatography followed by lyophilization. The salt obtained according to the procedure in said U.S. Patent is amorphous and hygroscopic, and therefore it is not suitable for a pharmaceutical product or is not easy to handle in the pharmaceutical preparations, in producing it on a commercial scale or in storage.

Therefore, there is a need for pure and stable crystalline salts of cefdinir, which are suitable for pharmaceutical preparations.

### SUMMARY OF THE INVENTION

We have now found that potassium salt of cefdinir can be obtained as a pure crystalline dihydrate, which can be prepared by a simple and efficient process. This crystalline salt may be conveniently formulated into tablets, suspensions, injectables and other pharmaceutical forms. Furthermore, it has been found that an efficient purification of cefdinir may be achieved by crystallizing it as cefdinir potassium dihydrate and then converting it to pure cefdinir.

### DETAILED DESCRIPTION OF THE INVENTION

The present invention provides a novel crystalline cefdinir potassium dihydrate of structural formula II. The characteristic IR and XRD spectra of cefdinir potassium dihydrate are given in FIGURE I and II respectively.

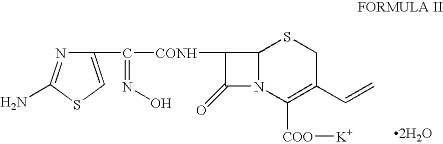

The present invention also provides a process for preparing cefdinir potassium dihydrate which comprises obtaining a solution of cefdinir potassium in a suitable solvent and crystallizing cefdinir potassium dihydrate from a solution thereof. The solution of cefdinir potassium can be obtained by adding a potassium salt of a weak acid to a suspension or solution of cefdinir in a suitable solvent The solution of cefdinir may be obtained either by dissolving cefdinir in a suitable solvent or directly from a reaction in which cefdinir is formed.

Often, when the potassium salt of a weak acid is added to a suspension of cefdinir in a suitable solvent the cefdinir potassium dihydrate starts crystallizing out even before cefdinir has gone into solution completely. Such a process is within the meaning of the process of the present invention.

Cefdinir used as the starting material may be obtained by any of the methods known in the prior art, for example, as described in U.S. Pat. Nos. 4,559,334; 4,870,168; 6,093,814; or as described in WO 92/7840, Japanese Patent applications 4/173781; 1/238587, and 2/000790 and are incorporated herein by reference.

The weak acid whose potassium salt may be used for forming potassium salt of cefdinir may be either an organic acid or an inorganic acid. Examples of suitable potassium salts include potassium acetate, potassium carbonate, potassium bicarbonate, and the like.

As per the present invention, the term “suitable solvent” may be any water miscible organic solvent in admixture with water. Suitable water miscible organic solvents include ketones such as acetone, ethylmethyl ketone; lower alcohols such as methanol, ethanol, propanol, isopropanol; nitriles such as acetonitrile; cyclic ethers such as tetrahydrofuran, dioxane, and mixture(s) thereof.

The crystallization may be performed at any suitable temperature depending on the solvent used. However, crystallization is preferably performed at about 0° C. to about 30° C., or preferably at about 5° C. to about 10° C.

In another aspect, the invention provides a process for the preparation of pure cefdinir which comprises preparing crystalline cefdinir potassium dihydrate of crude cefdinir, optionally recrystallized one or more times, and converting it to a free acid i.e. cefdinir. The product may be obtained as crystal A as described in U.S. Pat. No. 4,935,507, which is incorporated herein by reference. Alternatively, amorphous form of cefdinir similar to that produced by the method described in U.S. Pat. No. 4,559,334 may also be obtained via the purification process of the present invention.

The conversion of cefdinir potassium dihydrate to cefdinir can be accomplished by dissolving cefdinir potassium dihydrate in water and acidifying it to obtain the free acid, cefdinir.

“Crude cefdinir” is cefdinir prepared by any of the methods known in the prior art, which may contain anti-isomer, polymeric impurities or any other impurity which may arise during production or storage, such as degradation products. Crude cefdinir may be a solid or it may exist in a solvent e.g. in a mixture resulting directly from a reaction for the synthesis of cefdinir.

Cefdinir obtained by the process of the present invention has a purity greater than 99%.

In yet another aspect, the present invention also provides pharmaceutical compositions comprising cefdinir potassium dihydrate or cefdinir in combination with a pharmaceutical acceptable carrier and optionally included excipients, or diluents.

In the following section preferred embodiments are described by way of examples to illustrate the process of the invention. However, these are not intended in any way to limit the scope of the present invention.

Process for Preparing Cefdinir Potassium Dihydrate

### EXAMPLE 1

Potassium 7-(Z)-[2-(2-aminothiazol-4-yl)-2-hydroxyiminoacetamido]-3-vinyl-3-cephem-4-carboxylate, dihydrate.

To a suspension of cefdinir (5 g) in a mixture of water (25 ml) and acetone (25 ml) was added potassium acetate (1.75 g) at 25-30° C. The reaction mixture was stirred at this temperature for 2-3 hours for complete salt formation. The product started crystallizing out within about half an hour of potassium acetate addition. The reaction mixture was then cooled to 5-10° C. and stirred for 1.5 hours. The crystals were filtered, washed with acetone and dried to obtain 5.4 g of the title compound.
Yield 91%, HPLC purity: 99.85%, Water (w/w): 8.1%, K-content (w/w): 8.3%
   ′H- HMR (DMSO-d₆, 300 MHz): 11.4 (H, s), 9.42 (1 H, d, j=8.1 Hz), 7.16 (2 H, s), 6.99 (1 H, dd, j=11.1 Hz, 17.7 Hz), 6.64 (1 H, s), 5.6(1 H, dd, j=4.8 Hz, 8.1 Hz), 5.14 (1 H, d, j=17.7 Hz) 5.03(1 H, d,j=4.8 Hz), 4.93 (1 H, d,j=11.4 Hz), 3.4-3.8 (4 H, m,). IR (KBr, cm⁻¹): 3261, 1757, 1669, 1617, 1586.

### EXAMPLE 2

Potassium 7-(Z)-[2-(2-aminothiazol-4-yl)-2-hydroxyiminoacetamido]-3-vinyl-3-cephem-4-carboxylate, dihydrate

Cefdinir (5 g) was suspended in a mixture of water (25 ml) and isopropanol (25 ml) at 25-30° C. Potassium acetate (1.75 g) was added to this suspension and stirred for 2-3 hours for complete salt formation. The crystals were filtered, washed with acetone and dried to obtain 5.1 g of the title compound (Yield 86%, HPLC Purity: 99.5%).

Process for Preparing Pure Cefdinir

### EXAMPLE 3

Potassium 7-(Z)-[2-(2-aminothiazol-4-yl)-2-hydroxyiminoacetamido]-3-vinyl-3-cephem-4-carboxylate, dihydrate

Crude cefdinir (25 g, purity 94.5%) was suspended in a mixture of water (125 ml) and acetone (125 ml) at 25-30° C. Potassium acetate (8.75 g) was added to this suspension and stirred for 2-3 hours for complete salt formation. The crystals were filtered, washed with acetone and dried to obtain 22.5 g of the title compound (Yield 76%, HPLC Purity: 99.5%).

### EXAMPLE 4

7-(Z)-[2-(2-aminothiazol-4-yl)-2-hydroxyiminoacetamido]-3-vinyl-3-cephem-4-carboxylic acid (cefdinir)

Cefdinir potassium dihydrate (10 g) obtained from example 3 was dissolved in water (250 ml) at 30-35° C. Active carbon (1 g) and sodium metabisulfite (0.5 g) were added to the resulting solution and the mixture was stirred for 25-30 minutes at 30-35° C. It was filtered through celite and pH of the solution was adjusted to 2.4-2.6 at 30° C. and stirred at this temperature to obtain crystalline cefdinir (Yield 7.6 g, HPLC purity: 99.5%).

IR (KBr, cm⁻¹): 3295, 1767, 1683, 1622, 1519.

While the present invention has been described in terms of its specific embodiments, certain modifications and equivalents will be apparent to those skilled in the art and are intended to be included within the scope of the present invention.

1. Crystalline cefdinir potassium dihydrate of structural formula II.

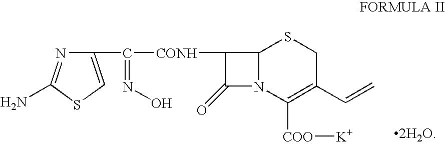

2. A process for the preparation of cefdinir potassium dihydrate of formula II 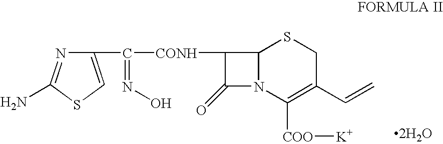
which comprises obtaining a solution of cefdinir potassium in a suitable solvent and crystallizing cefdinir potassium dihydrate from a solution thereof.

3. The process of claim 2 wherein the solution of cefdinir potassium is obtained by adding a potassium salt of a weak acid to a suspension or solution of cefdinir in said suitable solvent.

4. The process of claim 3 wherein the solution of cefdinir is obtained directly from a reaction in which cefdinir is formed.

5. The process of claim 2 wherein the potassium salt of a weak acid is selected from the group consisting of potassium acetate, potassium carbonate, and potassium bicarbonate.

6. The process of claim 2 wherein the suitable solvent is a water miscible organic solvent in admixture with water.

7. The process of claim 6 wherein the water miscible organic solvent comprises a ketone, lower alcohol, nitrile, cyclic ether and mixture(s) thereof.

8. The process of claim 7 wherein the ketone is acetone, ethyl methyl ketone and a mixture thereof.

9. The process of claim 7 wherein the lower alcohol is methanol, ethanol, propanol, isopropanol, and a mixture thereof.

10. The process of claim 7 wherein the nitrile is acetonitrile.

11. The process of claim 7 wherein the cyclic ether is tetrahydrofuran, dioxane and a mixture thereof.

12. The process of claim 2 further comprises converting crystalline cefdinir potassium dihydrate to cefdinir by treatment with an acid.

13. The process of claim 12 wherein the crystalline cefdinir potassium dihydrate is dissolved in water before treatment with an acid.

14. A process for the preparation of pure cefdinir which comprises preparing crystalline cefdinir potassium dihydrate of crude cefdinir, and converting it to cefdinir.

15. The process of claim 14, wherein the crude cefdinir is in solution which is obtained directly from a reaction for the synthesis of cefdinir.

16. Pure cefdinir prepared by the process of claim 14.

17. Cefdinir of claim 16 having a purity of more than 99%.

18. A pharmaceutical composition comprising cefdinir of claim 16 together with pharmaceutically acceptable carriers, excipients or diluents.

19. A pharmaceutical composition comprising cefdinir potassium dihydrate together with pharmaceutically acceptable carriers, excipients or diluents.
